(19) United States
(12) Patent Application Publication
(10) Pub. No.: US 2026/0199147 A1
(43) Pub. Date: Jul. 16, 2026

(54) MULTI-PANEL COMPOSITE DIAPER FOR USE IN CONJUNCTION WITH A HIP SPICA CAST

(71) Applicant: Daniel C. Taub, Groton, MA (US)

(72) Inventor: Daniel C. Taub, Groton, MA (US)

(21) Appl. No.: 19/279,226

(22) Filed: Jul. 24, 2025

Related U.S. Application Data
(60) Provisional application No. 63/675,015, filed on Jul. 24, 2024.

(51) Int. Cl. A61F 13/49; A61F 5/01

(52) U.S. Cl. CPC A61F 13/4906; A61F 5/01; A61F 2013/49098

(57) ABSTRACT

A composite diaper is configured for use in conjunction with a spica cast having cast interior and outer surfaces and a waste-elimination opening defined therein for facilitating the wearer's elimination of bodily waste. The composite diaper comprises mutually attached inner and outer diaper-panel sets. The inner diaper-panel set includes front and rear inner panels, and the outer diaper-panel set includes front and rear outer panels. The front and rear inner panels are configured for insertion through the waste-elimination opening and disposition between the cast interior surface and, respectively, the wearer's abdomen and buttocks. The front and rear outer panels are configured for drawing up and fastening to one another over the cast exterior surface, thereby retaining the inner diaper-panel set in position and, depending on the specific configuration, providing a secondary layer of bodily-waste absorbency and containment and/or a layer of protection against waste leakage to the cast exterior.

### PROVISIONAL PRIORITY CLAIM

Priority based on Provisional Application Ser. No. 63/675,015 filed Jul. 24, 2024 under the title “MULTI-PANEL DIAPER FOR USE IN CONJUNCTION WITH A HIP SPICA CAST” is claimed. Moreover, the entirety of the previous provisional application, including the drawings, is incorporated herein by reference as if set forth fully in the present application.

### BACKGROUND

The specific context and environment that inspired the present invention is the very challenging issue of dealing with diaper installation and removal on and from a person (e.g., an infant) wearing a type of body cast known as a “spica cast.” A spica cast—also called a “hip spica cast”—is an orthopedic cast designed to immobilize the cast wearer's thigh bones (femur bones) relative to their pelvis. A main, but not exclusive, application is to retain the hip joints in a proper position after corrective surgery for hip dysplasia, most commonly in infants and young children.

The cast is typically fabricated from fiberglass or plaster, and is provided with an interior lining to reduce discomfort by eliminating contact between the cast interior surface and the wearer's skin. A spica cast most commonly covers at least a portion of the torso including the abdomen and lower back, the waist, the pelvis, and at least a portion of each leg, but coverage can extend from the upper chest to below the knees.

The spica cast includes an opening in the groin area to permit the elimination, collection, and disposal of bodily waste. Presently, a known method for diapering a patient wearing a spica cast involves the use of two diapers of disparate sizes. One diaper—an “inner diaper”—is opened up and inserted up into the opening in the spica cast where the front and rear panels of the same are partially positioned between the spica cast and, respectively, the patient's abdomen and buttocks. However, it is not generally possible to fasten the inner diaper at the sides in accordance with usual diaper-installation methods due to the presence of the cast. Accordingly, a second, “outer diaper” is used and installed over the spica cast in a manner approximating as closely as practicable the normal installation of a diaper about a baby's hips, groin, and buttocks.

While the use of disparate inner and outer diapers as described above can be generally effective, the method is accompanied by certain complications and inconveniences. For example, caretakers must select and maintain an inventory of two sets of diapers, which are then separately installed. Moreover, once installed, the two separate diapers are free to move out of alignment with one another since they are not joined to one another.

Accordingly, a need exists for a single, multi-panel diaper including, preferably, four panels integrally formed as part of a unitary structure.

### SUMMARY

Variously embodied, a multi-panel composite diaper is configured for use in conjunction with a spica cast of a general type described in the background section of the present specification. The spica cast comprises a rigid shell defining mutually opposed cast interior and exterior surfaces and a waste-elimination opening for facilitating the wearer's elimination of bodily waste to the exterior of the cast. The cast interior surface defines a cast interior configured for accommodating a human cast wearer. Typically, the spica cast will also include an interior lining along the cast interior surface in order to reduce discomfort.

A first embodiment of a composite diaper has an inner diaper-panel set including a front inner panel and a rear inner panel. An outer diaper-panel set including a front outer panel and a rear outer panel is situated to the exterior of, and joined to, the inner diaper-panel set to define the composite diaper. The joined inner and outer diaper-panel sets are mutually oriented such that the front inner panel generally corresponds to the front outer panel and the rear inner panel generally corresponds to the rear outer panel. In at least one variant, the composite diaper is fabricated such that the inner diaper-panel set is a single, continuous panel structure and such that outer diaper-panel set is also a single, continuous panel structure. In this instance, each of the front and rear inner panels is a defined by a portion of the overall continuous structure defining the inner panel set, and each of the front and rear outer panels is likewise defined by a portion of the overall continuous structure defining the outer panel set.

The inner diaper-panel set is configured for at least partial disposition between the cast interior surface and the wearer's body, while the outer diaper-panel set is configured for contemporaneous disposition over the cast exterior surface. More specifically, the front and rear inner panels are configured for insertion and extension from the outer diaper-panel set through the waste-elimination opening for disposition on the cast interior between the cast interior surface and, respectively, the wearer's abdomen and buttocks. The front and rear outer panels are configured for drawing up and fastening to one another over a portion of the cast exterior surface. In a preferred configuration, the front and rear outer panels are fastened to one another over the cast exterior in the vicinity of the wearer's hips. In order to facilitate exterior fastening over the cast, the front outer panel has depending therefrom front-left and front-right fastener tabs that are cooperatively fastened to, respectively, rear-left and rear-right fastener tabs. Although the particular means by which the fastener tabs are fastened to one another is of secondary importance not central to the inventive concept, the fastener tabs preferably carry cooperatively couplable hook-and-loop fasteners of the general type ubiquitously sold under the trademark Velcro®.

Installation of the inner diaper-panel set between the cast interior surface and the wearer's body is in some respects analogous to the manner in which an inner diaper is installed in accordance with the existing method of using two separate diapers. However, because embodiments of the present invention are configured such that the inner and outer diaper-panel sets are structurally connected to one another, securement of the outer-diaper panel set over the cast exterior surface serves to retain the inner diaper panel set in place, while also facilitating limited adjustment of the inner panels through manipulation and adjustment of the outer panels.

While, in some embodiments, the panels of the inner diaper-panel set should be absorbent, it is not strictly necessary that the outer panels be absorbent since their function of retaining the inner panels in place within the cast does not depend on absorbency. However, it is preferred that the outer panels be configured to absorb and contain bodily waste (e.g., urine and fecal matter) and provide a secondary layer of protection against leaks to surfaces exterior to the cast and the composite diaper. Accordingly, it is to be understood that various combinations of absorbent and non-absorbent panels are possible within the scope and contemplation of the invention. For instance, neither, one, or both of the inner panels may be configured to absorb and contain bodily excretions, while, independently, neither, one, or both of the outer panels may be configured to absorb and contain bodily excretions.

Representative embodiments are more completely described and depicted in the following detailed description and the accompanying drawings.

### BRIEF DESCRIPTION OF THE DRAWINGS

FIG. 1 is a front view of a hip spica cast as worn by a human infant cast wearer;

FIG. 2 is a side view of a multi-panel composite diaper; and

FIG. 3 is a front view depicting a multi-panel composite diaper installed within and over a spica cast worn by a human infant.

### DETAILED DESCRIPTION

The following description of a variously configured multi-panel composite diaper is demonstrative in nature and is not intended to limit the invention or its application of uses. Accordingly, the various implementations, aspects, versions and embodiments described in the summary and detailed description are in the nature of non-limiting examples falling within the scope of the appended claims and do not serve to restrict the maximum scope of the claims as reasonably read in view of the specification. Additionally, throughout the specification and drawings, like elements across alternative embodiments, or various views of the same embodiment, are referenced by similar or identical numeric and/or alphanumeric reference characters, regardless of whether any particular element is called out explicitly in the specification with reference to a drawing in which a corresponding reference number may appear.

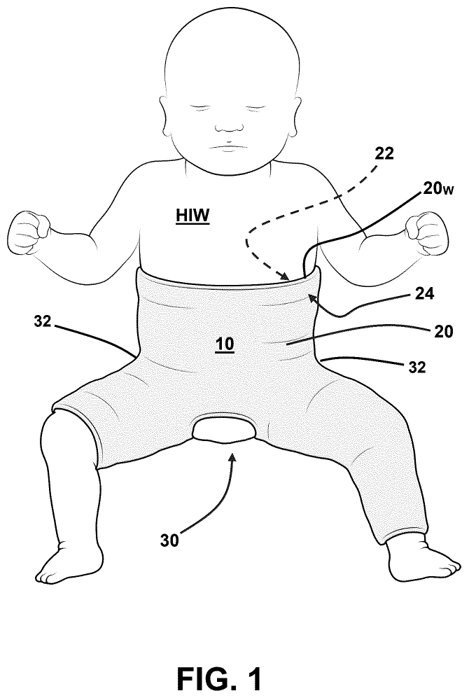

As explained in the summary, a common environment in which embodiments of the multiple-panel composite diaper will be employed is in conjunction with a hip spica cast. Accordingly, for contextual purposes, shown in FIG. 1 is an illustrative spica cast 10 disposed about a human infant wearer HIW. Individual body parts of the illustrative wearer HIW are not referenced by reference numbers because (i) they are not themselves a part of the embodied invention and (ii) such identification is not necessary since relevant parts of the human body are ubiquitously known. The spica cast 10 comprises a rigid shell 20 defined by a rigid-shell wall 20w having mutually opposed cast interior and exterior surfaces 22 and 24. In the vicinity of the wearer's groin, there is defined by and within the rigid-shell wall 20w a waste-elimination opening 30 for facilitating diapering and the wearer's elimination of bodily excretions (e.g., urine and feces).

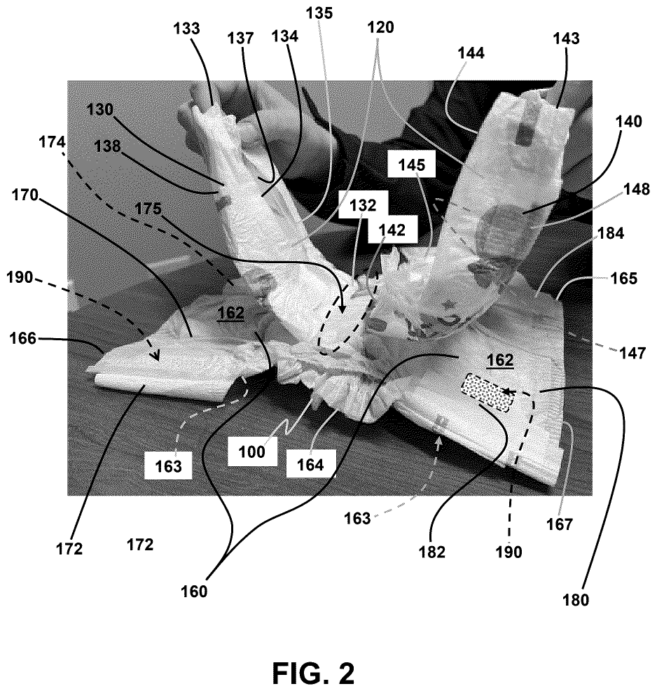

Referring now to FIG. 2, an illustrative composite diaper 100 has inner and outer diaper-panel sets 120 and 160. The inner diaper-panel set 120 includes front and rear inner panels 130 and 140, while the outer diaper-panel set 160 includes front and rear outer panels 170 and 180. The outer diaper-panel set 160 also has an outer-panel-set interior surface 162 and an outer-panel-set exterior surface 163 that share, and extend along, left and right outer-panel-set edges 164 and 165 which, in turn, extend fully between mutually opposed outer-panel-set front and rear edges 166 and 167.

The front and rear inner panels 130 and 140 are situated to the interior of, and attached to, the outer diaper-panel set 160 to define the composite diaper 100. More specifically, the front inner panel 130 has a front-panel base end 132, a front-panel distal edge 133 opposite the front-panel base end 132, and left and right front-inner-panel edges 134 and 135 that extend between the front-panel base end 132 and the front-panel distal edge 133. Additionally, the front inner panel 130 has mutually opposed front-inner-panel inner and outer surfaces 137 and 138. Similarly, the rear inner panel 140 has a rear-panel base end 142, a rear-panel distal edge 143 opposite the rear-panel base end 142, and left and right rear-inner-panel edges 144 and 145 that extend between the rear-panel base end 142 and the rear-panel distal edge 143. Moreover, analogously to the front inner panel 130, the rear inner panel 140 has mutually opposed front-inner-panel inner and outer surfaces 147 and 148.

The front inner panel 130 is attached to the outer-panel-set interior surface 162 at the front-panel base end 132, while the rear inner panel 140 is attached to the outer-panel-set interior surface 162 at the rear-panel base end 142. The panels of the inner and outer diaper-panel sets 120 and 160 are mutually positioned and oriented such that the front inner panel 130 generally corresponds to the front outer panel 170 and the rear inner panel 140 corresponds to the rear outer panel 180. Alternatively stated, the front and rear inner panels 130 and 140 are the panels of the inner diaper-panel set 120 that are most proximate to, respectively, the front and rear outer panels 170 and 180 of the outer diaper-panel set 160. Moreover, the front inner panel 130 is oriented such that the front inner-panel outer surface 138 is adjacent the outer-panel-set interior surface 162. Correspondingly, the rear inner panel 140 is oriented such that the rear inner-panel outer surface 148 is adjacent the outer-panel-set interior surface 162.

Described in the summary was one variant in which the composite diaper 100 is fabricated such that each of the inner and outer diaper-panel sets 120 and 160 is itself a single, continuous panel structure. It was further explained that, in such a case, each of the front and rear inner panels 130 and 140 is a defined by portion of the overall continuous structure defining the inner diaper-panel set 120, and that each of the front and rear outer panels 170 and 180 is likewise defined by a portion of the overall continuous structure defining the outer panel set 160. Such a variant is within the scope and contemplation of the claims, except to the extent that express claim limitations expressly negate the same. Moreover, in such an embodiment, the front-panel and rear-panel base ends 132 and 142 may be indistinguishable from one another (e.g., one and the same).

The version of FIG. 2 differs from that described in the preceding paragraph in that the front-panel and rear-panel base ends 132 and 142 of the front and rear inner panels 130 and 140, while both attached to the outer-panel-set interior surface 162 of the outer diaper-panel set 160, are separate from one another, or, more accurately, indirectly connected to one another by virtue of their individual attachment to the outer diaper-panel set 160. More specifically, in this example, while the front and rear outer panels 170 and 180 are defined portions of the overall continuous structure defining the outer diaper-panel set 160, the front and rear inner panels 130 and 140 are attached, independently of one another, to the outer-panel-set interior 162 of the continuous outer-diaper panel set 160. Moreover, the front and rear inner panels 130 and 140 are attached to the outer-panel-set interior surface 162 at locations spaced apart from one another such that there is exposed on the outer-panel-set interior surface 162, and between the front-panel and rear-panel base ends 132 and 142 of the front and rear inner panels 130 and 140, an outer-panel central portion 175 in the vicinity of where the front and rear outer panels 170 and 180 meet and transition one to the other. By this configuration, when the front and rear inner panels 130 and 140 are installed between the cast interior 22 and, respectively, the wearer's abdomen and buttocks, and the outer diaper-panel set 160 is secured about the cast exterior 24, bodily waste excreted from the wearer HIW can pass between the front and rear inner panels 130 and 140, through the waste-elimination opening 30 in the cast 10, and into the outer-panel central portion 175 of the outer diaper-panel set 160 that is aligned with the waste-elimination opening 30. While some bodily waste may be absorbed by one or both of the front and rear inner panels 130 and 140, allowing a substantial portion of the waste to pass through the waste-elimination opening 30 and into the outer diaper-panel set 160 contributes to cleanliness of the cast 10 and comfort of the wearer. With these goals in mind, it may even be desirable to configure at least one of the front and rear inner panels 130 and 140 from non-absorbent material, or at least from material less absorbent than the material from which the front and rear outer panels 170 and 180, including the central portion 175, are fabricated.

As previously indicated, the inner diaper-panel set 120 is configured for at least partial disposition between the cast interior surface 22 and the body of the wearer HIW, while the outer diaper-panel set 160 is configured for contemporaneous disposition over the cast exterior surface 24. More specifically, to install the composite diaper 100, the front and rear inner panels 130 and 140 are introduced through the waste-elimination opening 30 for disposition on the cast interior between the cast interior surface 22 and, respectively, the wearer's abdomen and buttocks. The front and rear outer panels 170 and 180 are then drawn up and fastened to one another over cast hip portions 28 of the cast exterior surface 24 in the vicinity of the hips of the wearer HIW. In order to facilitate exterior fastening over the cast 20, the front outer panel 170 has depending therefrom front-left and front-right fastener tabs 172 and 174 that are cooperatively fastened to, respectively, rear-left and rear-right fastener tabs 182 and 184 depending from the rear outer panel 180. The fastener tabs 172, 174, 182, and 184 preferably carry complementarily couplable hook-and-loop fasteners 190 of the general type ubiquitously sold under the trademark Velcro®. Alternative or additional illustrative means of fastening the fastener tabs 172, 174, 182, and 184 include pins, snaps, or ties.

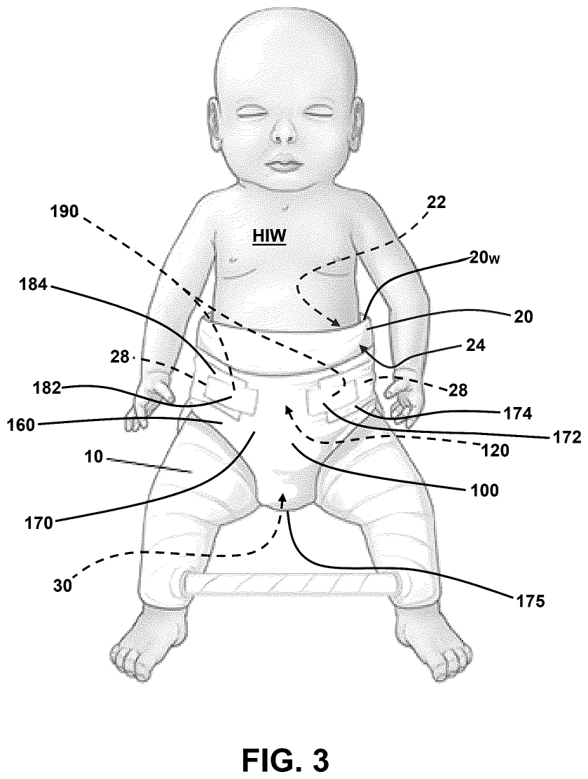

Depicted in FIG. 3 is an embodiment of the composite diaper 100 installed within and over a spica cast 10 worn by a human infant wearer HIW. With the composite diaper installed, the inner diaper-panel set 120 is entirely hidden from view (indicated by dashed lead arrow in FIG. 3) by the spica cast 100 and the outer diaper-panel set 160, rendering the composite diaper 100 in this case visually indistinguishable from an outer diaper used in association with the presently-common diapering practice using two diapers, as described in the background.

The foregoing is considered to be illustrative of the principles of the invention. Furthermore, since modifications and changes to various aspects and implementations will occur to those skilled in the art without departing from the scope and spirit of the invention, it is to be understood that the foregoing does not limit the invention as expressed in the appended claims to the exact constructions, implementations and versions shown and described.

What is claimed is:

1. A multi-panel composite diaper configured for use in conjunction with a spica cast having mutually opposed cast interior and cast exterior surfaces and a waste-elimination opening defined therein for facilitating the wearer's elimination of bodily waste, the composite diaper comprising:
an inner diaper-panel set including a front inner panel and a rear inner panel; and
an outer diaper-panel set including a front outer panel and a rear outer panel, the inner diaper-panel set being attached to the outer diaper-panel set to define the composite diaper, wherein
(a) the front and rear inner panels are configured for insertion through the waste-elimination opening for disposition between the cast interior surface and, respectively, the abdomen and buttocks of the wearer; and
(b) the front and rear outer panels are configured for drawing up and fastening to one another over a portion of the cast exterior surface.

2. The composite diaper of claim 1 wherein at least one of the inner panels is configured to absorb liquid.

3. The composite diaper of claim 2 wherein both the front and rear inner panels are configured to absorb liquid.

4. The composite diaper of claim 3 wherein, in addition to both the front and rear inner panels being configured to absorb liquid, at least one of the outer panels is configured to absorb liquid.

5. The composite diaper of claim 4 wherein
(i) the outer diaper-panel set has an outer-panel-set interior surface;
(ii) the front inner panel has a front-panel base end at which the front inner panel is attached to the outer-panel-set interior surface;
(iii) the rear inner panel has a rear-panel base end at which the rear inner panel is attached to the outer-panel-set interior surface; and
(iv) the front and rear inner panels are attached to the outer-panel-set interior surface at locations spaced apart from one another such that there remains exposed on the outer-panel-set interior surface, and between the front-panel and rear-panel base ends, an outer-panel central portion.

6. The composite diaper of claim 1 wherein
(i) the outer diaper-panel set has an outer-panel-set interior surface;
(ii) the front inner panel has a front-panel base end at which the front inner panel is attached to the outer-panel-set interior surface;
(iii) the rear inner panel has a rear-panel base end at which the rear inner panel is attached to the outer-panel-set interior surface; and
(iv) the front and rear inner panels are attached to the outer-panel-set interior surface at locations spaced apart from one another such that there remains exposed on the outer-panel-set interior surface, and between the front-panel and rear-panel base ends, an outer-panel central portion configured to be aligned with the waste-elimination opening in the spica cast and absorb bodily waste that passes therethrough.

7. A multi-panel composite diaper configured for use in conjunction with a spica cast having a shell defining cast interior and exterior surfaces and a waste-elimination opening for facilitating the elimination of bodily waste by the cast wearer to the exterior of the cast, the composite diaper comprising:
an outer diaper-panel set including a front outer panel, a rear outer panel, an outer-panel-set interior surface, and an absorbent central portion where the front and rear outer panels meet and transition one to the other;
an inner diaper-panel set including a front inner panel and a rear inner panel, the front and rear inner panels having, respectively, a front-panel base end and a rear-panel base end, the front-panel and rear-panel base ends being attached to the outer-panel-set interior surface at locations spaced apart from one another such that the absorbent central portion remains exposed between the front-panel and rear-panel base ends.

8. The composite diaper of claim 7 wherein at least one of the inner panels is configured to absorb and contain bodily waste.

9. The composite diaper of claim 8 wherein both the front and rear inner panels are configured to absorb and contain bodily waste.

10. The composite diaper of claim 7 wherein neither of the front and rear inner panels is configured to absorb bodily waste.

11. A combination spica cast and a multi-panel composite diaper comprising:
a spica cast having a shell defining a cast exterior surface, a cast interior surface defining a cast interior configured for accommodating a human cast wearer, and a waste-elimination opening for facilitating the elimination of bodily waste by the cast wearer to the exterior of the cast;
an outer diaper-panel set including a front outer panel, a rear outer panel, an outer-panel-set interior surface, and an absorbent central portion where the front and rear outer panels meet and transition one to the other; and an inner diaper-panel set including a front inner panel and a rear inner panel, the front and rear inner panels having, respectively, a front-panel base end and a rear-panel base end, the front-panel and rear-panel base ends being attached to the outer-panel-set interior surface at locations spaced apart from one another such that the absorbent central portion remains exposed between the front-panel and rear-panel base ends, wherein
(a) the front and rear outer panels are drawing up and fastened over a portion of the cast exterior surface with the absorbent central portion generally aligned with the waste-elimination opening; and
(b) the front and rear inner panels extend from the outer diaper-panel set to which they are attached and through the waste-elimination opening for disposition on the cast interior along the cast interior surface.

12. The composite diaper of claim 11 wherein at least one of the inner panels is configured to absorb and contain bodily waste.

13. The composite diaper of claim 12 wherein both the front and rear inner panels are configured to absorb and contain bodily waste.

14. The composite diaper of claim 11 wherein neither of the front and rear inner panels is configured to absorb bodily waste.
